(19) United States
(12) Patent Application Publication
(10) Pub. No.: US 2006/0063948 A1
(43) Pub. Date: Mar. 23, 2006

(54) Preparation of levulinic acid esters from alpha-angelica lactone and alcohols

(76) Inventor: Leo Ernest Manzer, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/088,115

(22) Filed: Mar. 23, 2005

Related U.S. Application Data
(60) Provisional application No. 60/556,235, filed on Mar. 24, 2004.

(51) Int. Cl. C07C 67/02

(52) U.S. Cl. 560/204; 549/263

(57) ABSTRACT

The present invention relates to a process for producing levulinic acid esters from α-angelica lactone and alcohols in the presence of a heterogeneous acid catalyst. In addition, a method is described herein for producing fuel or fuel additives comprising levulinic acid esters produced from α-angelica lactone and alcohol. In addition, compositions are described comprising levulinic acid esters for use as fuel or fuel additives.

### FIELD OF THE INVENTION

This invention relates to a process for producing levulinic acid esters from α-angelica lactone and alcohols in the presence of a heterogeneous acid catalyst. This invention also relates to the use of levulinic acid esters as fuel and fuel additives for gasoline fuel, diesel fuel and biofuel.

### BACKGROUND OF THE INVENTION

Levulinic acid esters are useful as solvents, plasticizers, flavoring agents and chemical intermediates. Levulinic acid esters also exhibit characteristics that make them particularly suitable for use as oxygenate additives for diesel fuel, gasoline fuel and biofuel, octane number-enhancing agents for gasoline, and as cetane number-enhancing agents in diesel fuels. The commercial use of levulinic acid esters has been limited due to the high cost of production. The production of levulinic acid esters from renewable resources, such as cellulosic biomass, however, represents a potentially low-cost route to the manufacture of these esters. The production of levulinic acid esters from biomass-derived levulinic acid is described, for example, in U.S. Patent Application 60/369,380 and “CL-2406”.

Langlois and Wolff (JACS (1948) 70:2624, U.S. Pat. No. 2,493,676) disclosed the production of pseudo esters of levulinic acid from α-angelica lactone and alcohol in the presence of an acid. They noted that secondary alcohols and reactive primary alcohols, such as allyl alcohol and benzyl alcohol, form pseudo esters readily, whereas normal (i.e., non-reactive) primary alcohols, notably the shorter chained alcohols such as methanol and ethanol, form normal (i.e., not pseudo) esters. A reaction of cyclohexanol and α-angelica lactone, for example, yielded 95% cyclohexyl pseudo ester. Langlois and Wolff stated that the rate of the reaction is controlled by the amount of catalyst used; if a large excess of acid such as hydrogen chloride is used at the start of the reaction, the resulting product is largely the normal ester, however by carefully adjusting the amount of acid it is possible to control the reaction without effecting an appreciable rearrangement of the pseudo ester to the normal ester. Acid (hydrogen chloride) concentrations used by Langlois and Wolff ranged from 0.1 mg to 1 g in a reaction volume of 50-100 ml.

Iwakura, et al. described the reaction of levulinic acid with ketene to produce α-angelica lactone. The authors noted that the addition of methanol or ethanol to the reaction product prior to distillation resulted in the formation of methyl or ethyl levulinate, respectively. The reaction of α-angelica lactone plus alcohol required an acid catalyst, such as the mineral acids sulfuric acid or hydrochloric acid. The authors also demonstrated the production of phenyl levulinate ester from 49 g α-angelica lactone (0.5 mol) and 0.1 mol phenol in 100 ml benzene in the presence of 0.1 g p-toluene sulfonic acid.

All known processes for producing levulinic acid esters from α-angelica lactone use corrosive, soluble mineral acids or environmentally unfriendly halogenated acids. Tightening environmental legislation is driving the chemical industry to alternative processes that do not use conventional mineral acids. The process of the present invention provides a novel, environmentally friendly method for producing levulinic acid esters with high rates of conversion and high selectivity using heterogeneous acids. The heterogeneous acid catalysts of the invention are advantageous in that they are easily separated from the reaction products, can be re-used in subsequent reactions, do not corrode reaction vessels or reactors, allow for synthesis by continuous flow, and have a reduced environmental impact.

### SUMMARY OF THE INVENTION

Described herein is a process for producing at least one levulinic acid ester from a reaction of α-angelica lactone with alcohol. The process comprises contacting α-angelica lactone with at least one alcohol in the presence of a heterogeneous acid catalyst: 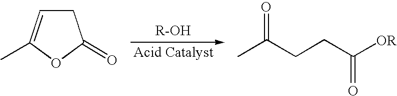
wherein:
(i) R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom; and
(ii) said at least one levulinic acid ester is optionally recovered.

The heterogeneous acid catalyst is selected from the group consisting of heterogeneous heteropolyacids, natural clay minerals, cation exchange resins, metal oxides, mixed metal oxides including zeolites, metal sulfides, metal sulfates, metal sulfonates, metal nitrates, metal phosphates, metal phosphonates, metal molybdates, metal tungstates, metal borates and combinations thereof, said metal being selected from elements from Groups 1, IIa, IIIa, VIIa, VIIIa, Ib and IIb of the Periodic Table of the Elements, aluminum, chromium, tin, titanium and zirconium.

The heterogeneous acid catalyst may comprise heterogeneous heteropolyacids of the general Formula XₐM_{b}O_{c}^{q-}, wherein X is selected from the group consisting of phosphorus, silicon, boron, aluminum, germanium, titanium, zirconium, cerium, cobalt and chromium; M is at least one transition metal selected from the group consisting of tungsten, molybdenum, niobium, vanadium, and tantalum; and q, a, b, and c are whole numbers or fractions thereof.

The heterogeneous acid catalyst may also be supported on a catalyst support, said catalyst support being selected from the group consisting of carbon, alumina, silica, silica-alumina, silica-titania, silica-zirconia, titania, titania-alumina, zirconia, barium sulfate, calcium carbonate, strontium carbonate, compounds thereof and combinations thereof.

The molar ratio of alcohol to α-angelica lactone is approximately 1:1 or greater than 1:1. The process of the invention is performed at a temperature of from about 1° C. to about 300° C., and a pressure of from about 0.1 MPag to about 15 MPag. Typically, the amount of catalyst used is from about 0.1% to about 50% by weight of the solution comprising the reactants.

The present invention also provides compositions comprising levulinic acid esters made by the process described above for use as fuels and fuel additives.

### DETAILED DESCRIPTION OF THE INVENTION

The present invention relates to a process for producing levulinic acid esters from α-angelica lactone and alcohols in the presence of an acid catalyst. In addition, a method is described herein for producing fuel additives comprising levulinic acid esters derived from α-angelica lactone and alcohol for use as oxygenates. Besides being used as oxygenate additives for diesel fuel, gasoline fuel and biofuel, the esters of the invention can also be used as octane number-enhancing agents for gasoline, and as cetane number-enhancing agents in diesel fuels. The reaction mixture of esters of the present invention can also be directly used as 100% fuel.

Definitions

In this disclosure, a number of terms and abbreviations are used. The following definitions are provided.

“Hydrocarbyl” refers to monovalent groups of atoms containing only carbon and hydrogen, and may be chiral or achiral. Unless otherwise stated, it is preferred in the method of the invention that hydrocarbyl (and substituted hydrocarbyl) groups contain 1 to 20 carbon atoms.

“Aliphatic” refers to a group of organic chemical compounds in which the carbon atoms are linked in open chains.

“Alkyl” refers to an alkyl group up to and including 20 carbons. Common examples of alkyl groups include methyl, ethyl, propyl, isopropyl, butyl, s-butyl, isobutyl, pentyl, neopentyl, hexyl, heptyl, isoheptyl, 2-ethylhexyl, cyclohexyl and octyl.

As used herein, the term “aryl” denotes aromatic cyclic groups including, but not limited to, phenyl groups. An “aromatic group” is benzene or compounds that resemble benzene in chemical behavior. Common examples of aryl groups include benzene, biphenyl, terphenyl, naphthalene, phenyl naphthalene, and naphthylbenzene.

A “heteroatom” is an atom other than carbon in the structure of a heterocyclic compound. A heterocyclic compound is a compound containing more than one kind of atom joined in a ring.

The “catalyst” useful in the invention is a substance that affects the rate of the reaction but not the reaction equilibrium, and emerges from the process chemically unchanged.

“Acid catalyst” refers to a substance which has the ability to donate protons as defined by Brönsted, or a substance which can form a covalent bond with an atom, molecule or ion that has an unshared electron pair as defined by Lewis. A further definition of acid catalysts and how to determine if a particular substance is acidic is explained in Tanabe, K., Catalysis: Science and Technology (Anderson, J. and Boudart, M. (eds.) (1981) Vol. 2, pages 232-273, Springer-Verlag, N.Y).

“Heterogeneous acid catalysts” or “solid acid catalysts” are acid catalysts used in heterogeneous acid catalysis. “Heterogeneous acid catalysis” refers to catalysis in which the catalyst constitutes a separate phase from the reactants and products.

“Zeolites” are naturally occurring or synthetic crystalline aluminosilicates that are composed of ordered arrangements of SiO₄ and AlO₄ tetrahedra kept neutral by ion-exchanged cations.

“Substituted” refers to a group attached to a reactant containing one or more substituent groups that do not cause the compound to be unstable or unsuitable for the use of reaction intended.

“Selectivity” refers to the weight percent of a particular reaction product in the total product weight (including the weight of unreacted reactants).

“Conversion” refers to the weight percent of a particular reactant that is converted to product.

“α-Angelica lactone” as used herein means a compound having the following formula:

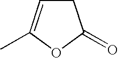

An “alcohol” of the invention is a compound having the Formula “R—OH” wherein R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom.

A “levulinic acid ester” of the invention is an ester having the formula exemplified below, wherein R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom:

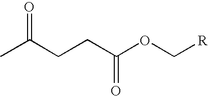

By the term “biofuel” is meant either a 100% biodiesel or a mixture comprising biodiesel and regular petroleum-based diesel from a refinery. For example, B20 is a mixture of 20% biodiesel based on vegetable oil, and 80% regular diesel. Biodiesel is a product of esterification of oils such as palm, canola, tallow, corn, and soy, with methanol.

By the term “octane number” is meant an empirical rating of the anti-knock quality of a fuel. “Knock” is caused by secondary ignition of fuel unburned after normal spark ignition, which gives rise to a fast moving flame front in an automobile's engine cylinder. Pressure waves are setup, which vibrate against the cylinder walls giving rise to a “knocking” sound. This feature of fuel is undesirable because it accelerates wear in the engine bearings and causes overheating in the cylinders. The tendency of the fuel to knock increases as the compression ratio increases. Certain fuels have better anti-knock characteristics than others because of their molecular structure, branched structures having better characteristics. On the arbitrary octane scale, iso-octane (C₈H₁₈) is given an octane value of 100; n-heptane (C₇H₁₆), is given a value of zero. The octane number of a fuel is determined by comparing its performance in a standard spark-ignition engine with the performance of various mixtures of iso-octane and n-heptane. The behavior of the fuel is carefully matched by a known mixture of iso-octane and n-heptane. The percentage of isooctane in this mixture is then taken as the octane number of the fuel.

The “cetane number” is used to evaluate fuels used in compression-ignition (diesel) engines and is analogous to octane number. Cetane (n-hexadecane, C₁₆H₃₄) is designated 100 and alpha-methyl-naphthalene (C₁₁H₁₀) as zero, so that the cetane number of a fuel is the proportion of the cetane in the mixture of these having the same ignition delay after injection of the fuel as the test fuel.

The invention described herein provides a process for preparing levulinic acid (or levulinate) esters from α-angelica lactone. The process comprises contacting α-angelica lactone with at least one alcohol in the presence of a heterogeneous acid catalyst: 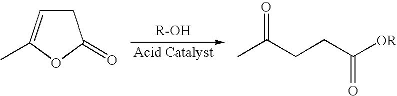
α-Angelica lactone for use in the process of the invention may be obtained by vacuum distilling levulinic acid as described in U.S. Pat. No. 2,809,203, or may be derived from biomass as described in U.S. Patent Application “CL-2406”.

In the present invention, R is an alkyl, aryl or alkaryl hydrocarbyl group having from one to twenty carbons, and R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom.

Typical alcohols of the invention are aliphatic alcohols having from one to ten carbons or aryl or alkaryl alcohols having from six to 13 carbons. Typical alcohols include, but are not limited to, methanol, ethanol, propanol, i-propanol, n-butanol, i-butanol, t-butanol, n-octanol, hexanol, cyclohexanol and benzyl alcohol. Mixtures of alcohols may also be used, resulting in a mixture of levulinic acid esters as the product.

In one embodiment of the invention, the molar ratio of alcohol to α-angelica lactone is greater than 1:1 (alcohol:α-angelica lactone); in another embodiment of the invention, the molar ratio of alcohol to α-angelica lactone is about 1:1.

In one embodiment, the temperature range for the process of the invention is from about 1° C. to about 300° C. In another embodiment, the temperature range for the reaction is from about 10° C. to about 200° C. In a further embodiment of the invention, the temperature range for the process of the invention is from about 80° C. to about 150° C.

In one embodiment, the pressure range for the reaction is from about 0.1 MPag to about 15 MPag. In another embodiment of the invention, the pressure range for the reaction is from about 0.1 MPag to about 5 MPag.

Catalysts useful in the process of the invention include heterogeneous (or solid) acid catalysts or combinations thereof; for a discussion of heterogeneous acid catalysts see Heterogeneous Catalyst Preparation, pages 215-258 (Hutchings, G. and Vedrine, J., in Basic Principles in Applied Catalysis, Baerns, M. (ed.), 2004 Springer-Verlag, New York), Solid Acid and Base Catalysts, pages 231-273 (Tanabe, K., in Catalysis: Science and Technology, Anderson, J. and Boudart, M (eds.) 1981 Springer-Verlag, New York) and Catalysis (Gates, B, in Kirk-Othmer Encyclopedia of Chemical Technology 2002 John Wiley & Sons, Inc. New York). Heterogeneous acid catalysts include, but are not limited to 1) heterogeneous heteropolyacids (HPAs), 2) natural clay minerals, such as those containing alumina or silica (including zeolites), 3) cation exchange resins, 4) metal oxides, 5) mixed metal oxides, 6) metal salts such as metal sulfides, metal sulfates, metal sulfonates, metal nitrates, metal phosphates, metal phosphonates, metal molybdates, metal tungstates, metal borates, and 7) combinations of groups 1-6. When present, the metal components of groups 4-6 may be selected from elements from Groups I, IIa, IIIa, VIIa, VIIIa, Ib and IIb of the Periodic Table of the Elements, as well as aluminum, chromium, tin, titanium and zirconium. Suitable heterogeneous acid catalysts include those having an H₀ of less than or equal to about 4.

Suitable HPAs include compounds of the general Formula XₐM_{b}O_{c}^{q-}, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof; for a discussion of HPAs see Hutchings, G. and Vedrine, J., supra, Pope, et al., Heteropoly and Isopoly Oxometallates: Inorganic Chemistry Concepts (Springer Verlag, New York, 1983) and Misono, et al. (Pure Appl. Chem. 2000 72:1305). Heteropolyacids are also known as “polyoxoanion”, “polyoxometallate” or “metal oxide cluster”. Structures of well known anions of HPAs include Keggin, Wells-Dawson and Anderson-Evans-Perloff structures. Examples of salts of HPAs include metal salts such as lithium, sodium, potassium, cesium, magnesium, barium, copper, gold and gallium, and onium salts such as ammonia, however, the invention is not limited thereto. Methods for preparing HPAs are well known in the art and are described, for example, in Hutchings, G. and Vedrine, J., supra; selected HPAs are also available commercially, for example, through Sigma-Aldrich Corp. (St. Louis, Mo.).

Examples of HPAs suitable for the process of the invention include tungstosilicic acid (H₄[SiW₁₂O₄₀].xH₂O), tungstophosphoric acid (H₃[PW₁₂O₄₀].xH₂O), molybdophosphoric acid (H₃[PMo₁₂O₄₀].xH₂O), molybdosilicic acid (H₄[SiMo₁₂O₄₀].xH₂O), vanadotungstosilicic acid (H_{4+n[SiV}ₙW₁₂₋ₙO₄₀].xH₂O), vanadotungstophosphoric acid (H_{3+n[PV}ₙW₁₂₋ₙO₄₀].xH₂O), vanadomolybdophosphoric acid (H₃₊ₙ[PVₙMo₁₂₋ₙO₄₀].xH₂O), vanadomolybdosilicic acid (H_{4+n[SiV}ₙMo₁₂₋ₙO₄₀].xH₂O), molybdotungstosilicic acid (H₄[SiMoₙW₁₂₋ₙO₄₀].xH₂O), molybdotungstophosphoric acid (H₃[PMoₙW₁₂₋ₙO₄₀].xH₂O), wherein n in the Formulas is an integer of 1 to 11 and x is an integer of 1 or more.

Natural clay minerals are well known in the art and are discussed in Tanabe, supra and Heterogeneous Catalysis in Industrial Practice, 2^{nd} Edition, pages 209-266 (C. Satterfield (1991) McGraw-Hill, Inc., New York). Examples include kaolinite, bentonite, attapulgite, montmorillonite and zeolites.

Suitable cation exchange resins are styrene-divinylbenzene copolymer-based strong cation exchange resins such as Amberlyst (Rohm & Haas; Philadelphia, Pa.), Dowex (for example, Dowex Monosphere M-31) (Dow; Midland, Mich.), CG resins from Resintech, Inc. (West Berlin, N.J.), and Lewatit resins such as MonoPluS™ S 100H from Sybron Chemicals Inc. (Birmingham, NJ).

Fluorinated sulfonic acid polymers can also be used as solid acid catalysts for the process of the present invention. These acids are partially or totally fluorinated hydrocarbon polymers containing pendant sulfonic acid groups, which may be partially or totally converted to the salt form. One particularly suitable fluorinated sulfonic acid polymer is Nafion® perfluorinated sulfonic acid polymer, (E.I. du Pont de Nemours and Company, Wilmington, Del.). One preferred form is Nafion® Super Acid Catalyst, a bead-form strongly acidic resin which is a copolymer of tetrafluoroethylene and perfluoro-3,6-dioxa-4-methyl-7-octene sulfonyl fluoride, converted to either the proton (H⁺), or the metal salt form.

Examples of suitable heterogeneous acids include, for example, zeolites such as CBV-3020 zeolite, fluorinated alumina, acid-treated silica, acid treated silica-alumina, acid-treated titania, acid-treated zirconia, sulfated-zirconia, heteropolyacids supported on zirconia, heteropolyacids supported on titania, heteropolyacids supported on alumina, heteropolyacids supported on silica, acid treated clays and combinations thereof.

Typically, the acid catalyst is used in an amount of from 0.1% to 50% by weight of the solution comprising the reactants. The acid catalysts employed herein may be used as powders, granules, or other particulate forms, or may be supported on an essentially inert support as is common in the art of catalysis. Selection of an optimal average particle size for the catalyst will depend upon such process parameters as reactor residence time and desired reactor flow rates.

The acid catalyst of the invention may be supported on a catalyst support. A supported catalyst is one in which the active catalyst agent is deposited on a support material by a number of methods, such as spraying, soaking or physical mixing, followed by drying, calcination, and if necessary, activation through methods such as reduction or oxidation. Materials frequently used as a support are porous solids with high total surface areas (external and internal) which can provide high concentrations of active sites per unit weight of catalyst. The catalyst support may enhance the function of the catalyst agent. A supported metal catalyst is a supported catalyst in which the catalyst agent is a metal.

Suitable supports include but are not limited to carbon, alumina, silica, silica-alumina, silica-titania, silica-zirconia, titania, titania-alumina, zirconia, barium sulfate, calcium carbonate, strontium carbonate, zeolites, compounds thereof and combinations thereof. The support can be neutral, acidic or basic, as long as the surface of the catalyst/support combination is acidic. In one embodiment of the invention, the support is selected from the group consisting of silica, titania, zirconia, silica-titania and silica-zirconia. Commonly used techniques for treatment of supports with metal catalysts can be found in B. C. Gates, Heterogeneous Catalysis (B. L. Shapiro (ed.) (1984) Vol. 2, pages 1-29, Texas A & M University Press, College Station, Tex.).

The process of the present invention may be carried out in batch, sequential batch (i.e., a series of batch reactors) or in continuous mode in any of the equipment customarily employed for continuous processes (see for example, H. S. Fogler, Elementary Chemical Reaction Engineering, Prentice-Hall, Inc., N.J., USA). Preferred reactors include trickle bed and catalytic distillation reactors.

It will be appreciated that the selectivities and yields of product may be enhanced by additional contact with the catalyst. For example, yields and selectivities may be increased where the reactor effluent containing a mixture of reactant and product may be contacted additional times over the catalyst under the reaction conditions set forth herein to enhance the conversion of reactant to product.

The process of the instant invention may additionally comprise the recovery or isolation of one or more of the levulinic acid esters. This can be done by any method known in the art, such as distillation, decantation, recrystallization, extraction or chromatography.

Compositions comprising levulinic acid esters produced by the process of the invention are useful as fuel additives. The levulinic acid esters may optionally be recovered as reaction products of the process of the invention for use as fuel additives, or the non-purified product mixture produced in the process of the invention, which may comprise unreacted alcohol, may be used directly. Besides being used as oxygenate additives for diesel fuel, gasoline fuel and biofuel, the esters of the invention can also be used as octane number-enhancing agents for gasoline, and as cetane number-enhancing agents in diesel fuels. The esters of the present invention can also be directly used as 100% fuel.

“Fuel additives” are substances that can improve the fuel efficiency of an engine, for example, as measured by the octane number, the cetane number or any other index suited to measure the efficiency of a particular fuel. A fuel additive may also perform the function of lubricating, cleaning and stabilizing the fuel and may improve performance, economy, and injector life, reduce emissions, reduce smoke related to an engine, help eliminate tank draining, lower a gel point of the fuel or provide a clean burning fuel that can inhibit polluting agents in emissions.

“Oxygenates” is a commonly referred to group of chemical compounds that raise the oxygen content of gasoline. Oxygen helps gasoline burn more completely, reducing harmful tailpipe emissions from motor vehicles. In one respect, the oxygen dilutes or displaces gasoline components such as aromatics (e.g., benzene) and sulfur. Additionally, it optimizes oxidation during combustion. Most gasoline suppliers meet the oxygen content requirements of the different clean fuel programs by adding oxygenate fuel additives, most commonly methyl tertiary-butyl ether (hereinafter referred to as MTBE), to gasoline blend stocks. Recently, various environmental protection agencies have begun raising concerns regarding the detection of MTBE in surface and ground water.

Levulinic acid esters of the invention with low water solubility can be used to meet governmental oxygen requirements for gasoline and oxy-gasoline fuels. These low solubility esters would have a reduced solubility in surface and subsurface water and could therefore reduce the impact on such waters from spills and emissions of oxygenated fuels. As shown in U.S. Patent Application 60/369,380, esters of the present invention, such as ethyl levulinate and methyl levulinate, have a significantly higher oxygen content than MTBE. Therefore, a lesser amount of esters is required to meet the various clean fuel programs' oxygen requirements for gasoline. Thus, the present invention provides compositions of levulinic acid esters or mixtures of levulinic acid esters produced by the process of the invention for use as fuel additives, such as oxygenates for gasoline, octane number-enhancing agents for gasoline, oxygenates for diesel, cetane number-enhancing agents for diesel or fuel additives for biofuel. The present invention also provides compositions of levulinic acid esters or mixtures of levulinic acid esters produced by the process of the invention for use as fuel.

As liquid organic based fuels for use in internal combustion engines, the reaction mixture containing levulinic acid esters or mixtures of levulinic acid esters as obtained from the process of the invention, can be used in the range of from about 1% to about 99% by volume, as additive to gasoline, diesel, or biofuel. A preferred range is from about 1% to about 90% by volume. A more preferred range is from about 1% to about 50% by volume. A further preferred range is from about 1% to about 20% by volume. Moreover, the reaction mixture comprising esters of the invention can also be used as 100% fuel.

The invention is further demonstrated by the following Examples.

### EXAMPLES

In the following examples, GC is gas chromatograph; the unit of pressure Mpag refers to MPa gage, and “temp” refers to temperature.

In the following examples Amberlyst 15 was obtained from Alfa Aesar (Ward Hill, Mass.); NAFION® was obtained from DuPont (Wilmington, Del.); CBV-3020E was obtained from PQ Corporation (Berwyn, Pa.).

### Example 1

General Procedure for the Reaction of Alcohols and α-Angelica Lactone

A 2 cc pressure vessel was charged with 700 mg of a solution consisting of alcohol, α-angelica lactone and 50 mg of a catalyst. The reactor was pressurized with nitrogen and heated if necessary to reactor temperature for a specified period of time. The vessel was then cooled, vented and the products analyzed by gas chromatography on a HP-6890 GC (Agilent Technologies; Palo Alto, Calif.) and HP-5972A GC-MS detector equipped with a 25 M×0.25 MM ID CP-Wax 58 (FFAP) column. The GC yields were obtained by adding methoxyethyl ether as the internal standard.

The examples described below were performed according to a similar procedure under the conditions indicated for each example.

### Examples 2-5

Reaction of α-Angelica Lactone (AGL) with Ethanol (EtOH) to Produce Ethyl Levulinate (EtLV)

| | | | | N₂ | | AGL | EtLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 2 | Amberlyst 15 | 1 | 100 | 5.52 | AGL (30%)/EtOH (70%) | 99.29 | 95.63 |
| 3 | 13% NAFION | 1 | 100 | 5.52 | AGL (30%)/EtOH (70%) | 99.44 | 88.98 |
| 4 | CBV-3020E | 1 | 100 | 5.52 | AGL (30%)/EtOH (70%) | 95.14 | 39.22 |
| 5 | Amberlyst 15 | 1 | 25 | 0.1 | AGL (30%)/EtOH (70%) | 75.21 | 59.23 |

### Examples 6-8

Reaction of α-Angelica Lactone (AGL) with 2-Propanol (2-PrOH) to Produce 2-Propyl Levulinate (2-PrLV)

| | | | | N₂ | | AGL | 2-PrLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 6 | Amberlyst 15 | 1 | 100 | 5.52 | AGL (30%)/2-PrOH (70%) | 97.45 | 72.85 |
| 7 | 13% NAFION | 1 | 100 | 5.52 | AGL (30%)/2-PrOH (70%) | 98.06 | 66.14 |
| 8 | CBV-3020E | 1 | 100 | 5.52 | AGL (30%)/2-PrOH (70%) | 95.14 | 45.74 |

### Examples 9-11

Reaction of α-Angelica Lactone (AGL) with 1-Octanol (1-OctOH) to Produce 1-Octyl Levulinate (1-OctLV)

| | | | | N₂ | | AGL | 1-OctLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 9 | Amberlyst 15 | 1 | 100 | 5.52 | AGL (30%)/1-OctOH (70%) | 99.48 | 98.66 |
| 10 | 13% NAFION | 1 | 100 | 5.52 | AGL (30%)/1-OctOH (70%) | 99.45 | 92.82 |
| 11 | CBV-3020E | 1 | 100 | 5.52 | AGL (30%)/1-OctOH (70%) | 96.27 | 68.73 |

### Examples 12-14

Reaction of α-Angelica Lactone (AGL) with 1-Hexanol (1-HxOH) to Produce 1-Hexyl Levulinate (1-HXLV)

| | | | | N₂ | | AGL | 1-HxLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 12 | Amberlyst 15 | 1 | 100 | 5.52 | AGL (30%)/1-HxOH (70%) | 98.51 | 98.36 |
| 13 | 13% NAFION | 1 | 100 | 5.52 | AGL (30%)/1-HxOH (70%) | 98.46 | 93.63 |
| 14 | CBV-3020E | 1 | 100 | 5.52 | AGL (30%)/1-HxOH (70%) | 95.00 | 65.98 |

### Examples 15-17

Reaction of α-Angelica Lactone (AGL) with t-Butanol (t-BuOH) to Produce t-Butyl Levulinate (t-BuLV)

| | | | | N₂ | | AGL | t-BuLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 15 | Amberlyst 15 | 1 | 100 | 5.52 | AGL (30%)/t-BuOH (70%) | 94.79 | 9.60 |
| 16 | 13% NAFION | 1 | 100 | 5.52 | AGL (30%)/t-BuOH (70%) | 96.91 | 11.87 |
| 17 | CBV-3020E | 1 | 100 | 5.52 | AGL (30%)/t-BuOH (70%) | 96.18 | 8.65 |

### Examples 18-23

Reaction of α-Angelica Lactone (AGL) with 1-Butanol (1-BuOH) to Produce 1-Butyl-Levulinate (1-BuLV)

| | | | | N₂ | | AGL | 1-BuLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 18 | Amberlyst 15 | 1 | 100 | 5.52 | AGL (30%)/1-BuOH (70%) | 99.80 | 98.60 |
| 19 | 13% NAFION | 1 | 100 | 5.52 | AGL (30%)/1-BuOH (70%) | 99.91 | 87.95 |
| 20 | CBV-3020E | 1 | 100 | 5.52 | AGL (30%)/1-BuOH (70%) | 98.42 | 61.78 |
| 21 | Amberlyst 15 | 1 | 25 | 0.1 | AGL (30%)/1-BuOH (70%) | 97.33 | 62.19 |
| 22 | 13% NAFION | 1 | 25 | 0.1 | AGL (30%)/1-BuOH (70%) | 47.57 | 12.92 |
| 23 | CBV-3020E | 1 | 25 | 0.1 | AGL (30%)/1-BuOH (70%) | 33.34 | 5.28 |

### Examples 24-26

Reaction of α-Angelica Lactone (AGL) with Benzyl Alcohol (BZOH) to Produce Benzyl Levulinate (BZLV)

| | | | | N₂ | | AGL | BzLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 24 | Amberlyst 15 | 1 | 100 | 5.52 | AGL (30%)/BzOH (70%) | 99.88 | 17.05 |
| 25 | 13% NAFION | 1 | 100 | 5.52 | AGL (30%)/BzOH (70%) | 99.62 | 63.98 |
| 26 | CBV-3020E | 1 | 100 | 5.52 | AGL (30%)/BzOH (70%) | 99.37 | 27.60 |

### Examples 27-29

Reaction of α-Angelica Lactone (AGL) with Methanol (MeOH) to Produce Methyl Levulinate (MeLV)

| | | | | N₂ | | AGL | MeLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 27 | Amberlyst 15 | 1 | 25 | 0.10 | AGL (30%)/MeOH (70%) | 85.34 | 55.86 |
| 28 | 13% NAFION | 1 | 25 | 0.10 | AGL (30%)/MeOH (70%) | 40.77 | 36.27 |
| 29 | CBV-3020E | 1 | 25 | 0.10 | AGL (30%)/MeOH (70%) | 46.05 | 23.48 |

### Examples 30-35

Reaction of α-Angelica Lactone (AGL) with Cyclohexanol (CvHxOH) to Produce Cyclohexyl Levulinate (CvHxLV)

| | | | | N₂ | | AGL | CyHxLV |
|---|---|---|---|---|---|---|---|
| Expt. | | Time | Temp | Pressure | | Conversion | Selectivity |
| No. | Acid Catalyst | (hrs) | (° C.) | (MPag) | Feedstock | (%) | (%) |
| 30 | Amberlyst 15 | 1 | 25 | 0.1 | AGL (30%)/CyHxOH (70%) | 84.98 | 87.85 |
| 31 | 13% NAFION | 1 | 25 | 0.1 | AGL (30%)/CyHxOH (70%) | 36.26 | 3.84 |
| 32 | CBV-3020E | 1 | 25 | 0.1 | AGL (30%)/CyHxOH (70%) | 38.97 | 4.69 |
| 33 | Amberlyst 15 | 1 | 100 | 5.5 | AGL (30%)/CyHxOH (70%) | 98.45 | 78.74 |
| 34 | 13% NAFION | 1 | 100 | 5.5 | AGL (30%)/CyHxOH (70%) | 98.42 | 72.65 |
| 35 | CBV-3020E | 1 | 100 | 5.5 | AGL (30%)/CyHxOH (70%) | 97.54 | 59.63 |

1. A process for preparing a reaction product comprising at least one levulinic acid ester, the process comprising contacting α-angelica lactone with at least one alcohol in the presence of a heterogenous acid catalyst: 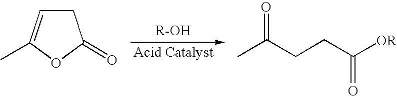
wherein:
(i) R is an alkyl, aryl or alkaryl hydrocarbyl group having from one to twenty carbons, and wherein R may be C₁-C₂₀ unsubstituted or substituted alkyl, C₂-C₂₀ unsubstituted or substituted alkenyl, C₂-C₂₀ unsubstituted or substituted alkynyl, C₃-C₂₀ unsubstituted or substituted cycloalkyl, C₃-C₂₀ unsubstituted or substituted cycloalkyl containing at least one heteroatom, C₆-C₂₀ unsubstituted or substituted aryl, C₆-C₂₀ unsubstituted or substituted aryl containing at least one heteroatom, C₇-C₂₀ unsubstituted or substituted alkaryl, or C₇-C₂₀ unsubstituted or substituted alkaryl containing at least one heteroatom; and
(ii) said at least one levulinic acid ester is optionally recovered.

2. The process of claim 1, wherein said heterogeneous acid catalyst is selected from the group consisting of heterogeneous heteropolyacids, natural clay minerals, cation exchange resins, metal oxides, mixed metal oxides including zeolites, metal sulfides, metal sulfates, metal sulfonates, metal nitrates, metal phosphates, metal phosphonates, metal molybdates, metal tungstates, metal borates and combinations thereof, said metal being selected from elements from Groups I, IIa, IIIa, VIIa, VIIIa, Ib and IIb of the Periodic Table of the Elements, aluminum, chromium, tin, titanium and zirconium.

3. The process of claim 2 wherein said heterogeneous catalyst has an Hₒ of less than or equal to about 4.

4. The process of claim 1 wherein said heterogeneous acid catalyst comprises heterogeneous heteropolyacids of the general Formula XₐM_{b}O_{c}^{q-}, wherein X is selected from the group consisting of phosphorus, silicon, boron, aluminum, germanium, titanium, zirconium, cerium, cobalt and chromium; M is at least one transition metal selected from the group consisting of tungsten, molybdenum, niobium, vanadium, and tantalum; and q, a, b, and c are whole numbers or fractions thereof.

5. The process of claim 1 wherein said heterogeneous catalyst is supported on a catalyst support, said catalyst support being selected from the group consisting of carbon, alumina, silica, silica-alumina, silica-titania, silica-zirconia, titania, titania-alumina, zirconia, barium sulfate, calcium carbonate, strontium carbonate, compounds thereof and combinations thereof.

6. The process of claim 5 wherein said heterogeneous catalyst is a heteropolyacid.

7. The process of claim 1 wherein the temperature of the reaction is from about 1° C. to about 300° C.

8. The process of claim 1 wherein the pressure of the reaction is from about 0.1 MPag to about 15 MPag.

9. The process of claim 1 wherein the content of the acid catalyst is from about 0.1% to about 50% by weight of the solution comprising the reactants.

10. The process of claim 1 wherein the temperature is from about 80° C. to about 150° C., the pressure is from about 0.1 to 5 MPag, and the heterogeneous acid catalyst is an ion exchange resin.

11. A composition comprising levulinic acid esters made by the process of claim 1.

12. The composition of claim 11 used as a fuel, an oxygenate for gasoline, an octane number-enhancing agent for gasoline, an oxygenate for diesel, a cetane number-enhancing agent for diesel or a fuel additive for biofuel.

13. A gasoline, diesel or biofuel comprising from 1% to 90% by volume of the composition of claim 11.

14. A gasoline, diesel or biofuel comprising from 1% to 50% by volume of the composition of claim 11.

15. A gasoline, diesel or biofuel comprising from 1% to 20% by volume of the composition of claim 11.

16. A process for manufacturing a fuel additive, the process comprising the process of claim 1.
